(19) United States
(12) Patent Application Publication
(10) Pub. No.: US 2002/0137780 A1
(43) Pub. Date: Sep. 26, 2002

(54) Differentiating agents for the treatment of inflammatory intestinal diseases

(76) Inventor: Gary W. Wu, Ardmore, PA (US)

Correspondence Address: LICATLA & TYRRELL P.C., 66 E. MAIN STREET, MARLTON, NJ, 08053, US

(21) Appl. No.: 09/896,984

(22) Filed: Jul. 2, 2001

Related U.S. Application Data
(63) Continuation-in-part of application No. 09/457,790, filed on Dec. 9, 1999.
Application No. 09/457,790 is a continuation-in-part of application No. 09/256,165, filed on Feb. 23, 1999, now abandoned.
Application No. 09/256,165 is a continuation-in-part of application No. 08/413,806, filed on Mar. 30, 1995.
Application No. 08/413,806 is a continuation-in-part of application No. 08/387,116, filed on Feb. 13, 1995.

(51) Int. Cl. A61K 31/557; A61K 31/426; A61K 31/16

(52) U.S. Cl. 514/369; 514/559; 514/549; 514/616

(57) ABSTRACT

A method for decreasing the inflammation associated with a chronic inflammatory intestinal condition in a patient is provided wherein the patient is administered an effective amount of a differentiating agent.

This application is a continuation-in-part of application Ser. No. 09/457,790, filed Dec. 9, 1999, which is a continuation-in-part of application Ser. No. 09/256,165, filed Feb. 23, 1999, which is a continuation-in-part of application Ser. No. 08/413,806, filed Mar. 30, 1995, issued as U.S. Pat. No. 5,981,597, which a continuation-in-part of application Ser. No. 08/387,116 filed Feb. 13, 1995, issued as U.S. Pat. No. 5,569,680.

### INTRODUCTION

This invention was supported in part by funds from the U.S. government (NIH Grant Nos. DK47709 and AI39368) and the U.S. government may therefore have certain rights in the invention.

### BACKGROUND OF THE INVENTION

Neoplastic disease is characterized by inappropriate cell proliferation relative to the rate of differentiation. A variety of agents which can induce transformed cells to express characteristics of a differentiated state and cease proliferating have been identified for use in the treatment of human cancers. Such agents include relatively simple polar/apolar compounds, retinoic acid and its derivatives, vitamin D₃ and its derivatives, tumor promoters, inhibitors of RNA or DNA synthesis including several agents used as cytotoxic therapy for cancers, growth factors, such as hematopoietic-cell growth factors, proteases, and hormones. The basic defect in cancers involves an imbalance in the relationship between proliferation of precursor cells and the differentiation of these cells. Differentiation factors can normalize the relationship of proliferation to differentiation. Consideration of a use for differentiating factors in the treatment of human cancers based largely upon in vitro studies that have demonstrated the effectiveness of these agents in inducing a wide variety of transformed cell lines to differentiate and stop growing. The successful treatment of human acute promyelocytic leukemia by retinoic acid established that cytodifferentiation therapy may have utility in the treatment of human malignancies. The clinical evaluation of additional differentiating agents has begun more recently.

Butyric acid has been shown to induce cytodifferentiation in vitro in a wide variety of neoplastic cells. Chen, Z. and Brutman, T. R. Cancer Res., 1994, 54:3494-3499. The potential utility of this agent as an antineoplastic has been limited, however, by the apparent difficulty in achieving effective concentrations of butyric acid in vivo. Chen and Brutman studied the effect of the prodrugs monobutyrin and tributyrin in vitro in inducing differentiation of human myeloid leukemia HL60 cells and murine erythroleukemia cells. Butyric acid, monobutyrin and tributyrin all induced erythroid differentiation of erythroleukemia. However, on a molar basis tributyrin was 3- to 4-fold more potent than butyric acid, whereas monobutyrin was much less potent than butyric acid. Based upon these experiments, it was suggested that tributyrin may be a promising candidate as a prodrug of butyric acid, either as a sole agent or in combination with other agents, for cytodifferentiation therapy of human leukemia and other malignancies and possibly for patients with hemoglobinopathies. Chen, Z. and Brutman, T. R., Cancer Res., 1994, 54:3494-3499.

Another differentiating agent, sodium phenylacetate, has been evaluated as an alternative to cytotoxic chemotherapy in the treatment of cancer. Phenylacetate has been demonstrated to promote maturation of various human leukemic cell lines. In addition, its use has been suggested in the treatment of prostate cancer. Samid et al., J. Clin. Invest., 1993, 91:2288-2295. Phenylacetate has been demonstrated to suppress tumor growth and promote differentiation in experimental models. Thibault et al., Can. Res., 1994, 54:1690-1694. It has also been shown that sodium phenylacetate and its precursor, sodium 4-phenylbutyrate, enhance fetal hemoglobin production in cultured erythroid progenitors derived from normal donors and patients with sickle cell anemia and β-thalassemia.

There also increasing evidence to suggest that 1,25-dihydroxy vitamin D₃ (1,25-(OH)₂D₃), or calcitrol, has important physiological effects on growth and differentiation in a variety of malignant and nonmalignant cell types. One of the earliest demonstrations of the antiproliferative effects of 1,25-(OH)₂D₃ was with the HL-60 human promyelocytic leukemia cell line. Treatment with physiological doses of 1,25(OH)₂D₃ suppressed cell growth and induced monocytic differentiation. Similar growth-inhibiting and differentiation-inducing effects have been demonstrated in vitro in other cell types including normal human bone cells, and in malignant cell lines derived from breast, malignant melanoma, histiocytic lymphoma and colon carcinoma. Halline et al., Endocrinology, 1994, 134(4):1710-1717.

Differentiating agents such as 1,25(OH)₂D₃ and analogs thereof, have also been used in the treatment of diseases related to disordered epidermal differentiation. In addition to producing vitamin D, epidermal cells (keratinocytes) make 1,25(OH)₂D₃, contain 1,25(OH)₂D₃, and respond to 1,25(OH)₂D₃ with changes in proliferation and differentiation. Bikle, D. D. and Pillai, S., Endocrine Reviews, 1993, 14(1):3-19. 1,25(OH)₂D₃ has been found to inhibit IL-la induced IL-8 production and mRNA expression in keratinocytes, fibroblasts and PBMC, but not in endothelial cells. Larsen et al., Biochem. Biophys. Res. Commun., 1991, 176(3) 1020-1026. Calcipotriol a vitamin D₃ analog which also inhibits cell proliferation and enhances cell differentiation. Calcipotriol has pharmacodynamic properties similar to those of calcitriol, the active metabolite of vitamin D₃. In several in vitro models both calcipotriol and calcitriol inhibit cell proliferation and enhance cell differentiation. Both drugs reduce cell numbers, total DNA content and incorporation of radiolabeled thymidine into DNA and increase the number of human keratinocytes with cornified envelopes and activity of enzyme-caused, protein crosslinking in the envelopes. In patients with psoriasis, calcipotriol also reduces dermal proliferation and enhances differentiation in lesional skin. Murdoch, D. and Clissold, S.P., Drugs, 1992, 43(3):415-429.

It has now been found that differentiating agents are also useful in the treatment of intestinal inflammatory diseases. Differentiating agents which alter the state of proliferation and ultimately the differentiation of colonic epithelial cells reduce the inflammation associated with intestinal diseases such as ulcerative colitis. One such class of differentiating agents is the peroxisome proliferators.

### SUMMARY OF THE INVENTION

The present invention provides methods of treating intestinal inflammatory disease, such as ulcerative colitis, in patients by administering an effective amount of one of a general class of compounds known as differentiating agents. These differentiating agents include a class of compounds that that are ligands that bind to and activate a class of nuclear hormone receptors known as Peroxisone Proliferator Activated Receptors (PPAR).

### BRIEF DESCRIPTION OF THE FIGURES

FIG. 1: A Northern blot of total RNA from preconfluent undifferentiated Caco-2 cells stimulated with IL-1β which hybridized with a cDNA probe for IL-8 and then rehybridized with a cDNA probe for 7S to demonstrate the loading and integrity of the RNA in each lane.

FIG. 2: A Northern blot of the same RNA samples as shown in FIG. 1 which hybridized using a probe for a fragment of the CCAAT/Enhancer Binding Protein delta isoform, an immediate gene product of the inflammatory response, and then rehybridized with a probe for 7S ribosomal RNA to demonstrate the loading and integrity of the RNA in each lane.

### DETAILED DESCRIPTION OF THE INVENTION

The normal mammalian small intestine lined by an epithelium that continuously renewed by proliferation of stem cells in intestinal crypts, migration of daughter cells from crypts onto villi, and extrusion of cells into the intestinal lumen at the tips of villi. Migration of enterocytes from crypts to villi coincident with the appearance of a differentiated phenotype. Accordingly, the intestinal mucosa represents a dynamic, complex epithelium that spatially segregated into a proliferating, undifferentiated compartment referred to as the crypts and a nonproliferating, differentiated compartment referred to as the villi. Gordon, J. I., J. Cell. Biol., 1989, 108:1187-1194. A similar compartmentalization occurs in the large intestine with undifferentiated proliferating epithelial cells located in the lower ⅔rds of the colonic crypts and undifferentiated cells located in the upper 3rd as well as on the surface mucosa. Risio, M., J. Cell. Biochem., 1992, 16G:79-87; Barnard et al., Gastroenterology, 1993, 105:67-73.

During intestinal inflammatory states, however, there an alteration in this pattern of epithelial differentiation. There an increase in epithelial proliferation with an expansion of cell populations in an undifferentiated state, referred to as crypt hypertrophy, as well as a decrease in cells exhibiting a differentiated phenotype, referred to as villus atrophy. This histologic pattern has been observed in many small intestinal inflammatory states such as celiac disease, pouchitis, tropical sprue and giardiasis. MacDonald, T. T., Ann. NY Acad. Sci., 1992, 664:202-209. A hyperproliferative state also occurs in the colonic epithelium during inflammatory states such as ulcerative colitis and parasitic infections. Risio, M., J. Cell. Biochem., 1992, 16G:79-87.

It believed that the expansion of the crypt cell compartment helps to perpetuate the intestinal inflammatory response. Indirect evidence suggests that the state of epithelial cell differentiation may determine whether or not intestinal epithelia are capable of responding to an inflammatory stimulus. Studies have shown that only undifferentiated proliferating crypt cells are capable of producing inflammatory cytokines such as IL-8 (Izzo et al., Gastroenterology, 1994, 106:A239) and IL-1β (Radema et al., Gastroenterology, 1991, 100:1180-1186). Furthermore, the production of neutrophil chemoattractive substances such as IL-8 exclusively by the crypt epithelium may explain why transmigration of neutrophils in acute intestinal inflammatory states occurs in the crypt compartment referred to as crypt abscesses. In addition, induction of MHC class 2 expression by the intestinal epithelium in response to interferon y or other inflammatory states such as GVDH or Trichinella spiralis infection occurs only in the proliferating crypt epithelium in both the small and large intestine. Bland, P., Immunology Today, 1988, 9:174-178.

The histologic appearance of active ulcerative colitis includes an intense lymphoplasmocytosis limited to the mucosa and submucosa which may be associated with a neutrophilic infiltrate invading the colonic epithelium, in particular the crypt abscess. This intense neutrophilic infiltrate observed with acute ulcerative colitis has been associated with elevated mucosal levels of interleukin-8 and circulating antibodies in patients with active ulcerative colitis as compared to normal controls and patients with active Crohn's disease. Hawkeyk et al., Agents Actions, 1992, 92:C23-26. Ulcerative colitis therefore categorized as a disorder of the colonic mucosa. Several investigators have shown that the colonic epithelium in a hyperproliferative state with expansion of the proliferative compartment from the lower crypt to the upper crypt extending to the surface epithelium. Biasco et al., Cancer Res., 1984, 44:5450-5454; Serafini et al., Gut, 1981, 22:648-652. This hyperproliferative state independent of the degree of inflammation and the duration of the disease, and exists even when the disease in an inactive state, thus suggesting an intrinsic abnormality of the colonic epithelium in ulcerative colitis. Biasco et al., Cancer Res., 1984, 44:5450-5454; Serafini et al., Gut, 1981, 22:648-652. Similar hyperproliferative states have been observed in patients at risk for colonic malignancy such as in familial polyposis coli, sporadic adenomas and familial nonpolyposis colon cancer. Risio, M., J. Cell. Biochem., 1992, 16G:79-87.

It has now been found that the ability of intestinal epithelial cells to respond to an inflammatory stimulus such as that resulting from ulcerative colitis dependent on the state of cell differentiation. It has been demonstrated that undifferentiated or pre-confluent Caco-2 cells can be stimulated by interleukin-1β (IL-1β) to produce interleukin-8 (IL-8) mRNA. In contrast, differentiated or post-confluent cells produce very little IL-8 mRNA after stimulation. It has also been found that nutrients and their derivatives, such as butyrate and vitamin D, which are capable of inducing differentiation, inhibit the expression of inflammatory mediators by intestinal epithelial cells. Induction of Caco-2 cell differentiation using differentiating agents such as sodium butyrate or vitamin D inhibits the expression of IL-8.

Butyrate enemas have been used to reduce inflammation in patients with distal ulcerative colitis. Breuer et al., Dig. Dis. Sci., 1991, 36:185-187; Scheppach et al., Gastroenterology, 1992, 103:51-56; Steinhart et al., Am. J. Gastro., 1994, 89:179-183. In two studies, butyrate enemas were shown to result in a significant clinical response in patients whose disease did not respond to traditional forms of treatment including use of corticosteroids and 5-amino salicylic acid compounds. The basis of this response unknown. Scheppach et al. observed that the labeling index of clonocytes in the upper crypt of patients with ulcerative colitis fell to that of normal healthy controls after treatment with butyrate enemas. However, the use of butyrate enemas severely limited due to its extremely strong odor which leads to patients refusing to continue treatment.

The metabolically active form of vitamin D, 1,25-dihydroxyvitamin D₃ [1,25-(OH)₂D₃] or calcitrol, has also been recognized as a differentiation agent. This form of vitamin D not only plays a critical role in calcium and phosphorus homeostasis, but also inhibits cell proliferation and induces differentiation of multiple malignant as well as non-transformed cell types. Pols et al., Clinical Endocrinology, 1994, 40:285-291.

In vitro data have now demonstrated that differentiating agents such as sodium butyrate, sodium propionate, 1,25-(OH)₂D₃, and phenylacetate are capable of inducing intestinal epithelial cell differentiation concurrent with inhibiting gene expression for the proinflammatory cytokine IL-8. Tributyrin, a less noxious prodrug of sodium butyrate, has also been shown to lead to an identical biological response in vitro at concentrations one third (⅓) that of sodium butyrate. These results were unexpected in light of in vitro experiments related to cytodifferentiation wherein it was found that tributyrin was only as effective as sodium butyrate in inhibiting cell proliferation at the same dose. Nudelman et al., J. Med. Chem., 1992, 35(4):687-694. Furthermore, these results suggest that colonic epithelial cells contain intracellular esterases that allow tributyrin to be metabolized to butyrate. No toxicity has been observed in mice treated with tributyrin either orally or intraperitoneally with a dose of 26.5 mmole/kg. Planlchon et al., J. Pharm. Sci., 1993, 82:1046-1048. In addition, tributyrin has been well tolerated in humans. For example, no detectable side effects were seen after six premature infants were fed butyrates for 4 days at doses of about 20 mmol/kg/day. Snyderman et al., Arch Dis. Child., 1955, 30:83-84.

In the present invention, a method is provided for decreasing the inflammation associated with a chronic inflammatory intestinal condition in a patient which comprises administering to a patient an effective amount of a differentiating agent. Examples of differentiating agents which can influence the inflammatory states of the intestine include, but are not limited to, polar/apolar compounds such as dimethyl sulfoxide and hexamethylene bisacetamide; retinoids such as 13-cis-retinoic acid, all-trans-retinoic acid and other analogs of retinol; vitamin D analogs including 1,25-(OH)₂D₃; histone hyperacetylators such as sodium butyrate and prodrugs thereof, sodium propionate and trichostatin A; hormones such as TGF-β and glucocorticoids; antioxidants such as PDTC; any compound that is a ligand for, and activates the PPAR family of nuclear hormone receptors such as, but not limited to, clofibrate, rezulin prostanoids and lipophillic compounds such as 15-deoxy-delta prostaglandin J2, polyunsaturated fatty acids, in particular fish oils, 13HODE and 15-HETE, a variety of non-steroidal anti-inflammatory drugs, and thiazolidinediones such as troglitazone, rosiglitazone maleate ((+)-5-[(4-[2-(methyl-2-pyridinylamino)ethoxy]-phenyl)methyl]-2,4-thiazolidine, (Z)-2-butenedioate(1:1)) and pioglitazone; and other miscellaneous differentiating agents such as phenylacetate and phenylbutyrate. By “effective amount” it meant a concentration sufficient to decrease the inflammation in the intestine associated with the condition. Effective concentrations of a differentiating agents can be easily determined based upon the data provided in the instant disclosure and knowledge of those of skill in the art concerning established dosing regimes for these agents for other therapeutic uses.

For example, retinal analogs have been studied for years. Four retinal analogues including retinal itself, tretinoin, etretinate, and isotretinoin have been administered orally for therapeutic purposes (Marcus, R. and A. M. Coulston, 1990, In: Goodman & Gilman's Pharmacological Basis of Therapeutics, pp. 1553-1563; Windhorst, D. B. and T. Nigra, 1982, J. Am. Acad. Dermatol., 6:675-682; Garry, P. J. et al., 1987, Am. J. Clin. Nutr., 46:989-994). These references teach specific doses of retinal analogs that can be used in humans, as well as the doses associated with toxicity in humans. With knowledge of the safe levels for human exposure and the levels known to have pharmacological activity, one of skill could routinely design a dosage regimen for administration of a retinol analog to humans which would be expected to decrease elevated levels of IL-8.

Sodium propionate has also been administered to humans safely (Scheppach, W. et al., Am. J. Gastroenterol., 1988, 83:850-853; Wolever, T. M. S. et al., Am. J. Gastroenterol., 1989, 84:1027-1033), as well as dimethyl sulfoxide which has been widely used in toxicology and pharmacology as a vehicle for compounds that are insoluble in water. In fact, dimethyl sulfoxide was approved by the U.S. Food and Drug Administration for use in humans for the treatment of interstitial cystitis in 1978. Phase I clinical studies have also been described for administration of phenylacetate (Thibault, A. et al., Cancer Res., 1994, 54:1690-1694; Thibault, A. et al., Cancer, 1995, 75:2932-2938). In addition, a number of studies have disclosed administration of hexamethylene bisacetamide to humans (Rowinsky, E. K. et al., Cancer Res., 1987, 47:5788-5795; Ward, F. T. et al., Cancer Res., 1991, 51:1803-1810; Andreeff, M. et al., Blood, 1992, 80:2604-2609; Conley, B. A. et al., Cancer Chemother. Pharmacol., 1992, 31:37-45) as well as peroxisome proliferators including clofibrate (Gustafson, A., Postgraduate Medical Journal, 1975, 51(8):66-71; Buchwald, H. Controlled Clinical Trials, 1982, 3:271-283) and rezulin (troglitazone; Nolan, J. J. et al., N. Engl. J. Med., 1994, 331:1188-93; Iwamoto, Y. et al., Diabetes Care, 1991, 14:1083-86). Thus, based upon the demonstrated pharmacological activity of differentiating agents herein and information available in the art regarding safe doses for these agents in humans, one of skill could routinely design treatment regimens for use in patients suffering from a chronic intestinal inflammatory condition.

By “patient” it meant an individual suffering from a chronic intestinal inflammatory condition. Examples of diseases or conditions which can be treated with the differentiating agents in accordance with the present invention include, but are not limited to, ulcerative colitis, Crohn's disease, Type A chronic gastritis, Type B chronic gastritis and graft vs. host disease (GVDH).

Once the inflammatory state has been initiated, an entire cascade of inflammatory mediators including cytokines, complement factors, prostaglandins, bradykinins and oxygen radicals follows. Accordingly, attempts to inhibit a single inflammatory pathway will not always lead to resolution of inflammation once the reaction has been initiated. An example of this the inability of IL-1 receptor antagonists to induce clinical remission in patients with active ulcerative colitis. In contrast, antiinflammatory agents such as glucocorticoids which inhibit multiple inflammatory pathways simultaneously, have been shown to be very effective in the treatment of inflammatory disease of the bowel. Thus, it believed that the administration of differentiating agents which inhibit the expression of inflammatory mediators by epithelial cells in conjunction with inhibitors of inflammatory mediators produced by immunocytes such as IL-1 receptor antagonist and cyclosporin, will result in a synergistic anti-inflammatory effect.

The following nonlimiting examples are provided to further illustrate the present invention.

### EXAMPLES

### Example 1

### Induction of IL-8 mRNA in Caco-2 Cells

Caco-2 cells (American Type Culture Collection, Rockville, Maryland) were plated at a density of 4×104 cells per cm² in 10 cm dishes containing Dulbecco's modified Eagle's medium (DMEM) with 10% fetal bovine serum and penicillin/streptomycin as described by Wu et al., J. Biol. Chem., 1992, 267:7863-7870 and Traber et al., Mol. Cell. Biol., 1992, 12:3614-3627. At days 5 (pre-confluent state) and 18 (post-confluent state) the cells were stimulated with complete medium containing 5 ng/ml of IL-1β (R and D Systems, Minneapolis, MN). Total RNA was isolated from these cells 2 and 4 hours after the addition of IL-1 containing medium as well as from a control cell population not treated with IL-1β in accordance with procedures described by Chomczynski, P. and Sacchi, N., Anal. Biochem., 1987, 162:156-159. Ten micrograms of RNA for each sample was electrophoretically separated, transferred to a nylon membrane, and UV crosslinked in accordance with procedures described by Traber et al., Am. J. Physiol., 1992, 262:G123-G130. A cDNA probe for IL-8 was prepared by RT-PCR of total RNA from Caco-2 cells stimulated with IL-1 for 2 hours. Random hexamers were used for reverse transcription of the RNA using reaction conditions similar to those described by Wu et al., Gastroenterology, 1993, 105:837-844. A 277 bp cDNA was amplified from the RT reaction by PCR using the 5′ primer IL-8(+103) and the 3′ primer IL-8(EX4), cloned using a TA-vector (Marchuk et al., Nuc. Acids Res., 1991, 19:1154), and labeled with ³²p using a Random Primers DNA labeling System (Gibco BRL, Gaithersburg, Md.). Hybridization of the Northern blots were performed using conditions as described by Huang et al., Mol. Endo., 1993, 7:1391-1398.

| | | |
|---|---|---|
| IL-8 (+103) | 5′-GTGGGATCCATGACTTCCAAGCTGGCC-3′ | (SEQ ID NO: 1) |
| IL-8 (EX4) | 5′-GTGGGATCCGAATTCTCAGCCCTCTTC-3′ | (SEQ ID NO: 2) |

GGATCC indicates the BAM HI site.

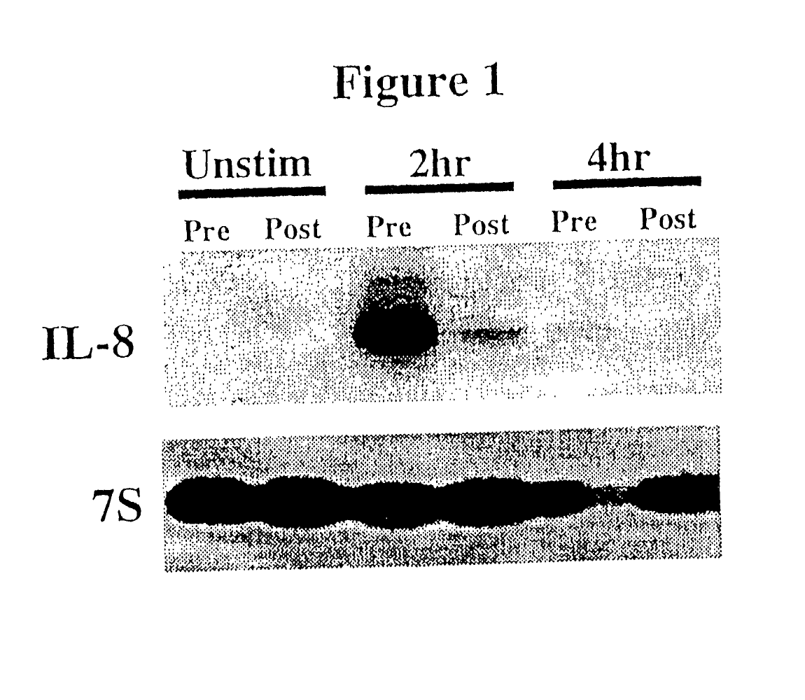

Results are Shown in FIG. 1.

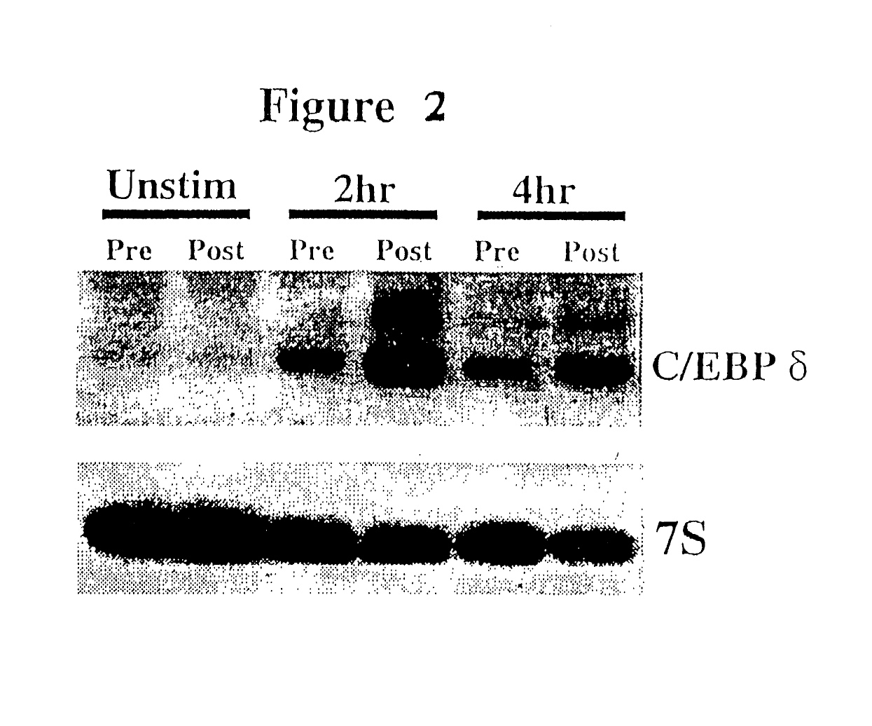

FIG. 2 shows a second Northern blot using the same total RNA hybridized with a 1.3 kb (Eco R1/Mlu1) restriction fragment of the CCAAT/Enhancer Binding Protein delta (C/EBPδ) isoform. Cao et al., Genes and Develop., 1991, 5:1538-1552. Both blots were subsequently stripped and rehybridized with a cDNA probe for 7S ribosomal RNA to demonstrate the loading and integrity of the RNA in each lane.

Stimulation of pre-confluent undifferentiated Caco-2 cells with IL-1βlead to a dramatic induction of IL-8 mRNA at 2 hours which rapidly decreased at late time points (see FIG. 1). In contrast, stimulation of post-confluent differentiated Caco-2 cells showed very minimal induction of IL-8 mRNA (see FIG. 1). Hybridization of this same RNA using a probe for C/EBPδ, an immediate gene product of the inflammatory response, demonstrated that post-confluent Caco-2 cells actually produce greater amounts of this mRNA than pre-confluent cells. Thus, although post-confluent Caco-2 cells are still capable of responding to an inflammatory status, it appears that the expression of a gene encoding an inflammatory modulating substance such as IL-8 differentially regulated. Furthermore, the C/EBPδ response of post-confluent cells suggests that functional IL-1 present in the medium of post-confluent Caco-2 cells and that functional IL-1 receptors as well as a signal transduction pathway must still exist in post-confluent Caco-2 cells.

### Example 2

### Inhibition of IL-1β Stimulated IL-8 Gene Expression with Differentiating Agents

Approximately 18 hours post-plating, Caco-2 cells were placed in complete medium containing various concentrations of sodium butyrate (Sigma Chemical Company, St. Louis, Mo.), sodium propionate (Sigma Chemical Company, St. Louis, Mo.), 1,25-(OH)₂D₃ (Biomol Research Laboratories, Plymouth Meeting, Pa.) or phenylacetate (Sigma Chemical Company, St. Louis, Mo.). Caco-2 cells were also plated that were not treated with any of these compounds. The medium was changed on a daily basis for 3 days. On day 5 all the cells (except for control) were stimulated with IL-1β (5 ng/ml) for 2 hours. Total RNA was then isolated and Northern blots performed as described in Example 1. The blots were hybridized to the IL-8 cDNA probe also described in Example 1.

Increasing concentrations of sodium butyrate from 0.1 to 2.5 mM led to a dose dependent reduction in steady state levels of IL-8 mRNA. These concentrations of sodium butyrate were well-tolerated by Caco-2 cells without any evidence of cell death. Treatment of Caco-2 cells with sodium butyrate at concentrations higher than 2.5 mM, which caused significant toxicity and cell death, led to increased expression of IL-8 mRNA. Concurrent with maximal inhibition of IL-8 mRNA expression at 2.5 mM the induction of mRNA for alkaline phosphatase, a marker for intestinal epithelial cell differentiation.

Sodium propionate (0.1 to 20 mM) also inhibited IL-8 mRNA expression in a dose dependent fashion concurrent with the induction of alkaline phosphatase mRNA expression. In contrast to sodium butyrate, however, treatment of Caco-2 cells with the maximum concentration of sodium propionate studied, 20 mM, did not lead to cell toxicity or death.

1,25-(OH)₂D₃ at concentrations of 10-8 and 10-9 also caused a decrease in steady state IL-8 mRNA expression. These concentrations of 1,25-(OH)₂D₃ have also been shown to inhibit Caco-2 cell proliferation and induce markers of intestinal epithelial differentiation. Halline et al., Endocrinology, 1994, 134:1710-1717.

Dose dependent inhibition of IL-8 mRNA expression was also observed with phenylacetate at concentrations ranging from 2.5 to 10 mM. There was no evidence of Caco-2 cell toxicity at any concentration of phenylacetate studied.

### Example 3

### Clinical Trial with the PPAR-γ Ligand

Fifteen patients with mild to moderately active ulcerative colitis (UC) were enrolled in an open-label prospective single-arm study of rosiglitazone given 4 mg orally twice daily for 12 weeks. Patients were evaluated at 0, 2, 4, 8 and 12 weeks for response to therapy and evidence of adverse reactions. Sigmoidoscopy was performed in each patient at the initial visit (0 weeks) and at 12 weeks.

The inclusion criteria were patients with documented UC on the basis of a combination of clinical, endoscopic, radiographic, and/or histological findings. Only patients with mild to moderately active UC despite a minimum of 4 weeks of therapy with at least 3 grams daily of an oral 5-ASA compound were eligible for inclusion. Disease activity was assessed using a standard Disease Activity Index well known in the art (Sandborn, W. J. et al. 1997. Ann. Intern. Med. 126:364-371). Mild to moderate disease on this index has a score of 4 to 10 inclusive. An index of 12 represents severe disease. Concomitant therapy with corticosteroids was permitted as long as the steroid dose was stable for a minimum of 4 weeks prior to enrollment. Concomitant therapy with azathioprine or 6-mercaptopurine was permitted as long as the patient had been receiving the medication for a minimum of 24 weeks and was on a stable dose for a minimum of 12 weeks prior to enrollment. No patients receiving concomitant therapy with rectally administered corticosteroids or 5-ASA compounds were allowed to enroll in the trial. Use of anti-diarrheal medications was also forbidden during the course of the trial. Exclusion criteria included evidence of infectious colitis, acute or chronic liver diseases, abnormal liver chemistries, heart failure, active malignancy other than skin cancer, diabetes mellitus requiring therapy, and a contraindication to flexible sigmoidoscopy or biopsy.

Patients with a final disease index score of 2 or less were considered to have achieved clinical remission during the study. A partial response to therapy was defined as a reduction in the index score of 2 points or more, but with a final score of 3 or greater. Endoscopic remission was defined as clinical remission plus a final score of 0 on the mucosal appearance subclass of the disease index. Since sigmoidoscopy was only performed at 0 and 12 weeks, a modified score was reported at the interim time points of 2, 4 and 8 weeks that included all components except endoscopic appearance of the bowel.

The results of treatment with rosiglitazone, a peroxisome proliferator, were that clinical remission was achieved in 4 of 15 patients (27%), with 3 of the 4 showing a final score of 0 which indicated endoscopic remission at 12 weeks. Of the remaining 11 patients, 4 of those achieved a partial response (27% of the 15). Therefore, of the initial 15 patients administered rosiglitazone, 8 of 15 (53%) had a significant clinical outcome, i.e., either clinical remission or partial response, despite previous failure of other therapies. Statistical analysis revealed that these data were statistically significant. Endoscopic remission was confirmed in 20% of the responders. At the conclusion of the 12 week trial, 6 of the 8 responders continued to received rosiglitazone, with all but one of these patients continuing to do well after a median follow-up of 29 weeks (range 16-41 weeks). Only a single patient that was in remission at the conclusion of the trial that discontinued therapy had a subsequent relapse 8 weeks later.

In addition, of the patients that were receiving corticosteroids at the start of the study (8 of 15), at the conclusion of the study 6 of the 8 had reduced their daily dose by 50% or more.

Only 4 of 15 patients in the trial reported serious adverse events during the study. Two patients required hospitalization for worsening disease activity. One patient developed nephrotic syndrome (proteinuria) approximately 8 weeks into the therapy and therapy was withdrawn. Another patient developed severe Group A streptococcal pharyngitis and aeseptic arthritis during the final week of therapy. The patient was hospitalized but with treatment with antibiotics and corticosteroids his condition resolved. As this patient's UC had responded to treatment (partial responder), he elected to continue the study medication after the trial. None of the patients developed abnormal liver chemistries during the course of the study and only one patient developed a mild, transient lower extremity edema.

A comparison of published data where placebo control groups have been included in studies of mild to moderate UC indicated that typical placebo response rates were 26.7% as compared to the current response rate of more than 53%. These data, therefore, indicate that this class of drugs, which are ligands that activate the PPAR family of nuclear hormone receptors, represent a new therapy alternative for ulcerative colitis.

What is claimed is:

1. A method for decreasing inflammation associated with a chronic inflammatory intestinal condition in a patient comprising administering to a patient an effective amount of a differentiating agent.

2. The method of claim 1 wherein the differentiating agent comprises a ligand for the peroxisome proliferator activated receptor family of nuclear hormone receptors.

3. The method of claim 2 wherein the differentiating agents are clofibrate, prostanoids, 15-deoxy-delta prostaglandin J2, fish oils, 13HODE, 15-HETE, non-steroidal anti-inflammatory drugs, or thiazolidinediones.

4. The method of claim 2 wherein the differentiating agent is rosiglitazone.
